(19) United States
(12) Patent Application Publication
(10) Pub. No.: US 2026/0043117 A1
(43) Pub. Date: Feb. 12, 2026

(54) Experimental system and method for high-temperature oxidation and quenching of cladding materials under reactor severe accident

(71) Applicant: XI'AN JIAOTONG UNIVERSITY, Xi'an, Shaanxi (CN)

(72) Inventors: Yapei Zhang, Xi'an (CN); Pengfei Ma, Xi'an (CN); Xiaocheng Wu, Xi'an (CN); Xiurui Li, Xi'an (CN); Guanghui Su, Xi'an (CN); Suizheng Qiu, Xi'an (CN); Wenxi Tian, Xi'an (CN)

(21) Appl. No.: 19/362,343

(22) Filed: Oct. 18, 2025

(30) Foreign Application Priority Data
Oct. 18, 2024 (CN) 202411461594.6

(51) Int. Cl. C22F 1/00; C22F 1/18; F27B 17/02; F27D 7/02; F27D 11/02; F27D 19/00; F27D 99/00; G01N 33/20; G21C 3/07; H05B 3/00

(52) U.S. Cl. CPC C22F 1/002; C22F 1/186; F27B 17/02; F27D 7/02; F27D 11/02; F27D 99/0006; G01N 33/20; H05B 3/0038; F27D 2019/0009; F27D 2019/0018; G21C 3/07; H05B 2203/032

(57) ABSTRACT

An experimental system for high-temperature oxidation and quenching of cladding materials under reactor severe accident includes: a gas supply system, a heating section, a cooling system, and a rapid quenching system. The gas supply system supplies mixed gas of steam and argon. The heating section includes an infrared radiation furnace and a quartz glass tube. The rapid quenching system includes a constant-temperature water tank, high-temperature resistant hoses, quenching quartz glass tube, and movable rails. At a reaction zone, samples and atmosphere can be heated up to 1400° C. at an ultra-high heating rate exceeding 100° C./s under reactive atmospheres such as steam, and the sample is subjected to rapid quenching after high-temperature steam oxidation testing. The experimental provides ultra-high heating rates and rapid quenching, which facilitates the reach on micro- and macro-mechanisms of high-temperature reactions and quenching in materials.

### CROSS REFERENCE OF RELATED APPLICATION

The present invention claims priority under 35 U.S.C. 119(a-d) to CN 202411461594.6, filed Oct. 18, 2024.

### BACKGROUND OF THE PRESENT INVENTION

### Field of Invention

The present invention relates to a technical field of high-temperature oxidation and quenching experimental performance testing of structural materials, and more particularly to an experimental system and method for studying the high-temperature steam oxidation and quenching behavior of cladding materials under reactor severe accident.

### Description of Related Arts

Nuclear fuel assemblies serve as the energy source for nuclear power plants, which constitute the core components of reactors. Due to superior mechanical properties, corrosion resistance, and neutron economic efficiency, zirconium alloys have been used in China's operational and under-construction light water reactors after decades of technological refinement and operational feedback. These assemblies employ UO₂ as fuel and Zr alloys as cladding. However, during the 2011 Fukushima nuclear accident in Japan, Zr alloy cladding underwent rapid oxidation, generating significant hydrogen gas and heat. This led to core meltdown and hydrogen explosions, causing severe environmental and societal impacts. Consequently, the nuclear industry has focused on the inherent limitations of UO₂—Zr alloy fuel.

To enhance the inherent safety of fuel assemblies, the concept of ATF (Accident Tolerant Fuel) was proposed and has become a hot research topic all over the world. To apply ATF in commercial light water reactors, scholars worldwide have conducted extensive theoretical and experimental research, focusing on developing and selecting novel cladding materials. The most promising candidate for commercial application is Cr-coated Zr cladding. However, during beyond-design-basis accidents, reactor core temperatures may exceed the eutectic temperature of the Cr—Zr alloy. Therefore, within the CEA-Framatome-EDF joint nuclear fuel program, the concept of “EATF (Enhanced Accident Tolerant Fuel)” for LWRs (light water reactors) was introduced. To expand the database and better understand EATF's response time and temperature, research has been extended to lower and higher oxidation temperatures, including “DEC (Design Extension Conditions)” up to 1500° C. Existing studies indicate that eutectic reactions between the chromium coating and zirconium substrate will occur within a temperature range of 1300-1330° C. The temperature at which Cr—Zr eutectic reactions occur in the Cr-coated Zr alloy cladding depends on the preparation method and thickness of the coating. Following the eutectic reaction, the liquid phase formed between the Cr coating and Zr significantly accelerates the consumption rate of the Cr coating, potentially causing rapid loss of oxidation resistance. Research on the potential impact of this reaction on high-temperature oxidation behavior and associated embrittlement and failure mechanisms is conventionally in its infancy. No suitable experimental system has yet been developed to reasonably control the heating rates in high-temperature steam environments and to achieve the rapid quenching after oxidation to simulate the accident conditions of cladding.

### SUMMARY OF THE PRESENT INVENTION

To address the above issues in prior art, an object of the present invention is to provide an experimental system and method for high-temperature oxidation and quenching of cladding materials under reactor severe accident, which introduce steam for high-temperature oxidation and quenching. By precisely controlling heating rates and target temperatures, the high-temperature oxidation quenching behavior of cladding under accident conditions and subsequent mechanical properties can be simulated.

Accordingly, in order to accomplish the above object, the present invention provides:
an experimental system for high-temperature oxidation and quenching of cladding materials under reactor severe accident, comprising: a first argon cylinder (1) and a second argon cylinder (2), which are connected in series to a main argon pipeline via a first valve (101), a second valve (102) and corresponding pipelines that are externally arranged, wherein a first thermocouple (201) and a first flowmeter (401) are installed on the main argon pipeline; a steam generator (3) communicates with an external deionized water pipeline via a third valve (103); a second thermocouple (202) and a first pressure sensor (301) serve as temperature and pressure detection devices for the steam generator (3), and a first water level gauge (501) serves as a water level detection device for the steam generator (3); a fourth valve (104) is configured to perform steam bypass discharge; steam is fed into the argon main pipeline through a fifth valve (105) and a second flowmeter (402) to be mixed with argon gas at a preset ratio; a steam temperature is measured by a third thermocouple (203); a gas main pipeline is connected to a vacuum pump (4) via a sixth valve (106); an inlet (6) of a heating quenching device is connected to a mixed gas pipeline via a static gas mixer (5) and a seventh valve (107); mixed gas in the heating quenching device is uniformly mixed by the static gas mixer (5); the mixed gas pipeline is equipped with heating wires for temperature control of the mixed gas; the mixed gas pipeline is also equipped with a second pressure sensor (302) and a fourth thermocouple (204); the heating quenching device comprises an infrared radiation furnace (12), a constant-temperature water tank (10), a high-temperature resistant hose (9), a quartz glass tube (13), a sealing ring (20), a quenching quartz glass tube (8), an upper rail slider fixture (16), a lower rail slider fixture (7), a slide rail bracket (17), and a chiller (11), wherein the mixed gas enters the quartz glass tube (13) within the infrared radiation furnace (12) through the inlet (6); a cladding sample is suspended within an infrared focused heating zone (21) at a center of the quartz glass tube (13); the mixed gas is discharged through an outlet (14) of the heating quenching device; a gold-plated reflective surface within the infrared radiation furnace (12) is cooled via the chiller (11), and a pipeline flow rate of the chiller (11) is monitored by a third flowmeter (403), which is then controlled by an eighth valve (108); a height of the cladding sample within the infrared focused heating zone (21) is adjusted via the upper rail slider fixture (16) on an upper portion of the slide rail bracket (17) to achieve uniform heating; a bottom end of the quartz glass tube (13) is opened or closed by the sealing ring (20) located at the bottom end; the lower rail slider fixture (7) at a lower portion of the slide rail bracket (17) clamps the quenching quartz glass tube (8), thereby performing vertical movement of the quenching quartz glass tube (8) within the quartz glass tube (13) for rapid quenching after high-temperature oxidation testing of the cladding sample; a heating temperature of the cladding sample is collected via a fifth thermocouple (205) which is fast in response and exposed, and a heating temperature sequence of the cladding sample is collected through a data acquisition system (18) connected to the exposed fifth thermocouple (205); an infrared radiation furnace temperature control system (19) is connected to the infrared radiation furnace (12) for temperature control of the cladding sample during high-temperature steam oxidation testing.

Preferably, the infrared radiation furnace (12) employs four high-power tungsten filament infrared lamps as heat sources, and heating elements of the infrared lamps are sealed within quartz glass; a stainless steel surface is process with gold plating for reflection and focusing of short-wave infrared radiation; the quartz glass tube (13) is located at a center of the infrared radiation furnace.

Preferably, the slide rail bracket (17) automatically controls vertical movement of the upper rail slider fixture (16) and the lower rail slider fixture (7).

Preferably, constant-temperature water is provided by the constant-temperature water tank (10); water is exchanged between the quenching quartz glass tube (8) and the constant-temperature water tank (10) via the high-temperature resistant hose (9) for temperature control and movement; rapid quenching of the cladding sample after the high-temperature oxidation testing is achieved through automated control of rapid movement of the quenching quartz tube (8).

Preferably, the infrared radiation furnace temperature control system (19) employs a PID (Proportion Integral Differential) algorithm to maintain a constant heating rate, thereby achieving temperature control of the cladding sample during the high-temperature steam oxidation testing.

Preferably, the infrared radiation furnace (12) is capable of heating to 1400° C. with a heating rate exceeding 100° C./s under steam conditions.

Preferably, the data acquisition system (18) comprises a data acquisition card, a measurement module, a signal conditioner and a computer-driven software module, wherein the data acquisition card is connected to the fifth thermocouple (205) via a junction box.

Preferably, the cladding sample is suspended by a platinum-rhodium wire (15) within the infrared focused heating zone (21) at the center of the quartz glass tube (13); wherein the height of the cladding sample within the infrared focused heating zone (21) is adjusted by clamping the platinum-rhodium wire (15) with the upper rail slider fixture (16) on the upper portion of the slide rail bracket (17), thereby achieving uniform heating.

An experimental method for high-temperature oxidation and quenching of cladding materials under reactor severe accident is provided, comprising steps of: performing high-temperature oxidation testing on the cladding materials in a steam environment, then performing rapid quenching to obtain mechanical properties of the cladding materials; wherein the experimental method comprises specific steps of: before testing, keeping all valves closed; using a high-precision electronic balance to measure a mass of the cladding sample multiple times and calculating an average value; opening a third valve (103) to introduce deionized water into a steam generator (3) until a preset water level is reached, and then closing the third valve (103); opening a sixth valve (106) and a vacuum pump (4) to evacuate an experimental pipeline, then closing the sixth valve (106); supplying argon gas from a first argon cylinder for testing, with a second argon cylinder (2) serving as a backup; opening a first valve (101) to introduce the argon gas for purging air from the experimental pipeline and from a quartz glass tube (13); opening a fifth valve (105) to feed steam generated by the steam generator (3) into a main argon gas pipeline; activating a static gas mixer (5) and opening a seventh valve (107) to uniformly mix the argon gas and the steam; determining a steam temperature in a mixed gas pipeline by adjusting heating wires and monitoring a temperature sensed by a fourth thermocouple (204); closing a sealing ring (20), so that mixed gas formed by the argon gas and the steam flows upwards through the quartz glass tube (13) and exits through an outlet (14); connecting the cladding sample to an upper rail slider fixture (16) using a platinum-rhodium wire (15), and activating the upper rail slider fixture (16) on a slide rail bracket (17) to move the cladding sample to a bottom of an infrared focused heating zone (21); activating the infrared radiation furnace (12) for heating with a preset heating rate and a target temperature; activating a chiller (11) to cool a stainless steel gold-plated reflective wall of the infrared radiation furnace (12); activating a data acquisition system (18) to collect temperature information of the cladding sample using a fifth thermocouple (205) which is fast in response and exposed, and transmitting the temperature information to an infrared radiation furnace temperature control system (19), thereby controlling a heating rate and a heating temperature of the infrared radiation furnace; just before high-temperature steam oxidation ends, opening the sealing ring (20) at a bottom end of the quartz glass tube (13), and activating a lower rail slider fixture (7) of the slide rail bracket (17) to move a quenching quartz glass tube (8) to the bottom of the infrared focused heating zone (21); at an instant the high-temperature steam oxidation ends, deactivating the infrared radiation furnace (12) while simultaneously activating the lower rail slider fixture (7) to lift the quenching quartz glass tube (8), thereby rapidly quenching the cladding sample after the high-temperature oxidation testing; then activating the upper rail slider fixture (16) of the slide rail bracket (17) to lift and remove the cladding sample, and sequentially closing all pipeline valves and deactivating all experimental instruments;

after testing, measuring the mass of the tested cladding sample using the high-precision electronic balance and calculating the average value; preparing a cross-sectional sample from the cladding sample using a metallographic preparation material, and characterizing oxidation behavior via EDS (Energy Dispersive Spectroscopy), SEM (Scanning Electron Microscopy), or TEM (Transmission Electron Microscopy); subjecting the cladding sample to circumferential compression testing at a preset displacement rate using a circumferential compression testing machine, so as to obtain a stress-strain curve of the cladding sample after quenching, thereby obtaining an offset strain of the cladding sample to characterize the mechanical properties.

Compared to conventional heating technologies, the experimental heating system of the present invention offers the following advantages:
(1) High-precision sample temperature control: by integrating the infrared radiation furnace with the temperature control system, the heating rate and heating temperature of the cladding sample can be precisely regulated; additionally, cooling rate and temperature holding at arbitrary temperatures can be accurately controlled.
(2) High-speed heating and cooling: the high-energy-density infrared radiation lamps and the gold-plated reflective walls enable an ultra-high heating rate for heating.
(3) Clean heating: the heating elements of the infrared lamps are sealed within the quartz glass, eliminating contamination from element gases; furthermore, the infrared furnace uses no insulation materials, preventing dust and gas contamination compared with resistance furnaces.
(4) Heating and cooling in various atmospheres: heating and cooling can be performed under vacuum, high-purity inert gas, or static or flowing steam conditions; the operation is straightforward, utilizing heating and cooling chambers made of quartz glass that allow infrared transmission.

Compared with prior art, the experimental quenching system of the present invention offers the following advantages: the designed quenching system achieves rapid quenching after high-temperature steam oxidation testing through precise control of the upper and lower rail slider fixtures of the slide rail bracket, minimizing the exposure time of the cladding sample to air. By lifting the quenching quartz glass tube, the present invention can avoid the impact caused by directly immersing the cladding sample into water, as seen in prior art, which enhances the authenticity of the simulated high-temperature steam oxidation and quenching testing, facilitating a clearer understanding of the residual mechanical properties of the cladding materials after high-temperature steam oxidation and quenching under severe accidents.

### BRIEF DESCRIPTION OF THE DRAWINGS

FIG. 1 is a sketch view of an experimental system for high-temperature oxidation and quenching of cladding materials under reactor severe accident according to the present invention; and

FIG. 2 illustrates experimental processes of an infrared radiation furnace and a quenching system in the experimental system of the present invention.

### DETAILED DESCRIPTION OF THE PREFERRED EMBODIMENT

Technical solutions according to an embodiment of the present invention will now be described in detail with reference to the accompanying drawings.

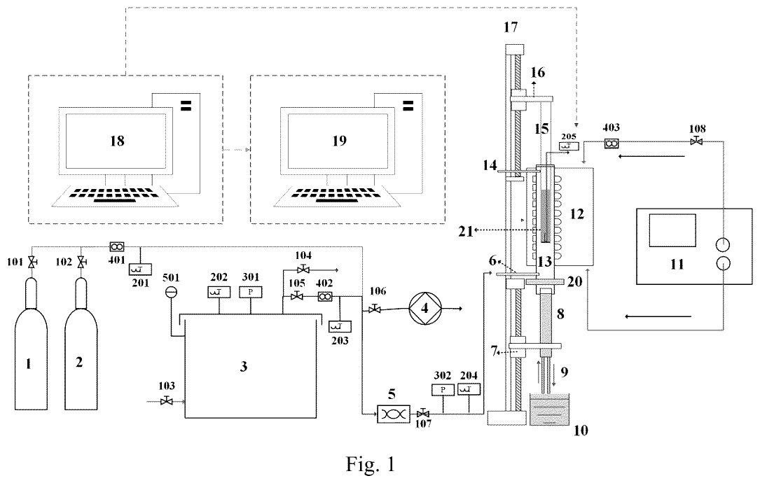

Referring to FIG. 1 of the drawings, an experimental system for high-temperature oxidation and quenching of cladding materials under reactor severe accident comprises: a gas supply system, a heating section, a cooling system, and a rapid quenching system. The gas supply system supplies mixed gas of steam and argon. The heating section includes an infrared radiation furnace, a platinum-rhodium wire and a quartz glass tube. The rapid quenching system includes a constant-temperature water tank, high-temperature resistant hoses, a quenching quartz glass tube, and movable rails. In the gas supply system, a first argon cylinder 1 and a second argon cylinder 2 are provided, which are connected in series to a main argon pipeline via a first valve 101, a second valve 102 and corresponding pipelines that are externally arranged, wherein a first thermocouple 201 and a first flowmeter 401 are installed on the main argon pipeline; a steam generator 3 communicates with an external deionized water pipeline via a third valve 103; a second thermocouple 202 and a first pressure sensor 301 serve as temperature and pressure detection devices for the steam generator 3, and a first water level gauge 501 serves as a water level detection device for the steam generator 3; a fourth valve 104 is configured to perform steam bypass discharge; steam is fed into the argon main pipeline through a fifth valve 105 and a second flowmeter 402 to be mixed with argon gas at a preset ratio; a steam temperature is measured by a third thermocouple 203; a gas main pipeline is connected to a vacuum pump 4 via a sixth valve 106; an inlet 6 of a heating quenching device is connected to a mixed gas pipeline via a static gas mixer 5 and a seventh valve 107; mixed gas in the heating quenching device is uniformly mixed by the static gas mixer 5; the mixed gas pipeline is equipped with heating wires for temperature control of the mixed gas; the mixed gas pipeline is also equipped with a second pressure sensor 302 and a fourth thermocouple 204; the heating quenching device comprises an infrared radiation furnace 12, a constant-temperature water tank 10, a high-temperature resistant hose 9, a quartz glass tube 13, a sealing ring 20, a quenching quartz glass tube 8, an upper rail slider fixture 16, a lower rail slider fixture 7, a slide rail bracket 17, a chiller 11, and a platinum-rhodium wire 15, wherein the mixed gas enters the quartz glass tube 13 within the infrared radiation furnace 12 through the inlet 6; a cladding sample is suspended within an infrared focused heating zone 21 at a center of the quartz glass tube 13 by the platinum-rhodium wire 15; the mixed gas is discharged through an outlet 14 of the heating quenching device; a gold-plated reflective surface within the infrared radiation furnace 12 is cooled via the chiller 11, and a pipeline flow rate of the chiller 11 is monitored by a third flowmeter 403, which is then controlled by an eighth valve 108; a height of the cladding sample within the infrared focused heating zone 21 is adjusted via the upper rail slider fixture 16 on an upper portion of the slide rail bracket 17 to achieve uniform heating; a bottom end of the quartz glass tube 13 is opened or closed by the sealing ring 20 located at the bottom end; the lower rail slider fixture 7 at a lower portion of the slide rail bracket 17 clamps the quenching quartz glass tube 8, thereby performing vertical movement of the quenching quartz glass tube 8 within the quartz glass tube 13 for rapid quenching after high-temperature oxidation testing of the cladding sample; a heating temperature of the cladding sample is collected via a fifth thermocouple 205 which is fast in response and exposed, and a heating temperature sequence of the cladding sample is collected through a data acquisition system 18 connected to the exposed fifth thermocouple 205; an infrared radiation furnace temperature control system 19 is connected to the infrared radiation furnace 12 for temperature control of the cladding sample during high-temperature steam oxidation testing.

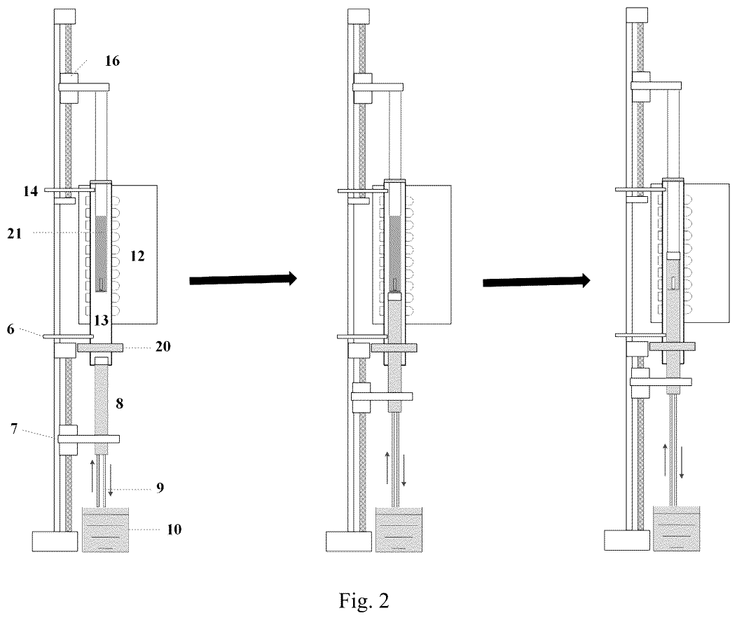

Referring to FIG. 2, constant-temperature water in the heating quenching device is provided by the constant-temperature water tank 10, and the bottom end of the quartz glass tube 13 is opened or closed by the sealing ring 20; water is exchanged between the quenching quartz glass tube 8 and the constant-temperature water tank 10 via the high-temperature resistant hose 9 for temperature control and movement; rapid quenching of the cladding sample after the high-temperature oxidation testing is achieved through automated control of rapid movement of the quenching quartz tube 8. During both the high-temperature oxidation and quenching phases, the infrared radiation furnace 12 remains active, which heats the cladding sample within the infrared focused heating zone. The height of the quenching quartz glass tube 8 is adjusted by the lower rail slider fixture 7 to prepare for rapid quenching. As long as the oxidation testing ends, the infrared radiation furnace 12 is immediately deactivated. As shown in FIG. 2, the infrared focused heating zone of the quenching phase now disappears, wherein the lower rail slider fixture 7 is rapidly adjusted to move the quartz glass tube 8 upwards, thereby performing rapid quenching of the cladding sample.

An experimental method for high-temperature oxidation and quenching of cladding materials under reactor severe accident is provided, comprising steps of: before testing, keeping all valves closed; using a high-precision electronic balance to measure a mass of the cladding sample multiple times and calculating an average value; opening a third valve 103 to introduce deionized water into a steam generator 3 until a preset water level is reached, and then closing the third valve 103; opening a sixth valve 106 and a vacuum pump 4 to evacuate an experimental pipeline, then closing the sixth valve 106; supplying argon gas from a first argon cylinder for testing, with a second argon cylinder 2 serving as a backup; opening a first valve 101 to introduce the argon gas for purging air from the experimental pipeline and from a quartz glass tube 13; opening a fifth valve 105 to feed steam generated by the steam generator 3 into a main argon gas pipeline; activating a static gas mixer 5 and opening a seventh valve 107 to uniformly mix the argon gas and the steam; determining a steam temperature in a mixed gas pipeline by adjusting heating wires and monitoring a temperature sensed by a fourth thermocouple 204; closing a sealing ring 20, so that mixed gas formed by the argon gas and the steam flows upwards through the quartz glass tube 13 and exits through an outlet 14; connecting the cladding sample to an upper rail slider fixture 16 using a platinum-rhodium wire 15, and activating the upper rail slider fixture 16 on a slide rail bracket 17 to move the cladding sample to a bottom of an infrared focused heating zone 21; activating the infrared radiation furnace 12 for heating with a preset heating rate and a target temperature; activating a chiller 11 to cool a stainless steel gold-plated reflective wall of the infrared radiation furnace 12; activating a data acquisition system 18 to collect temperature information of the cladding sample using a fifth thermocouple 205 which is fast in response and exposed, and transmitting the temperature information to an infrared radiation furnace temperature control system 19, thereby controlling a heating rate and a heating temperature of the infrared radiation furnace; just before high-temperature steam oxidation ends, opening the sealing ring 20 at a bottom end of the quartz glass tube 13, and activating a lower rail slider fixture 7 of the slide rail bracket 17 to move a quenching quartz glass tube 8 to the bottom of the infrared focused heating zone 21; at an instant the high-temperature steam oxidation ends, deactivating the infrared radiation furnace 12 while simultaneously activating the lower rail slider fixture 7 to lift the quenching quartz glass tube 8, thereby rapidly quenching the cladding sample after the high-temperature oxidation testing; then activating the upper rail slider fixture 16 of the slide rail bracket 17 to lift and remove the cladding sample, and sequentially closing all pipeline valves and deactivating all experimental instruments; after testing, measuring the mass of the tested cladding sample using the high-precision electronic balance and calculating the average value; preparing a cross-sectional sample from the cladding sample using a metallographic preparation material, and characterizing oxidation behavior via EDS, SEM, or TEM; subjecting the cladding sample to circumferential compression testing at a preset displacement rate using a circumferential compression testing machine, so as to obtain a stress-strain curve of the cladding sample after quenching, thereby obtaining an offset strain of the cladding sample to characterize the mechanical properties.

The foregoing details provide a further explanation of the present invention based on specific principles. However, the implementation of the present invention should not be limited to these descriptions. For those skilled in the art, simple derivations or substitutions made without departing from the underlying concept of the present invention should be considered within the protection scope thereof.

What is claimed is:

1. An experimental system for high-temperature oxidation and quenching of cladding materials under reactor severe accident, comprising: a first argon cylinder (1) and a second argon cylinder (2), which are connected in series to a main argon pipeline via a first valve (101), a second valve (102) and corresponding pipelines that are externally arranged, wherein a first thermocouple (201) and a first flowmeter (401) are installed on the main argon pipeline; a steam generator (3) communicates with an external deionized water pipeline via a third valve (103); a second thermocouple (202) and a first pressure sensor (301) serve as temperature and pressure detection devices for the steam generator (3), and a first water level gauge (501) serves as a water level detection device for the steam generator (3); a fourth valve (104) is configured to perform steam bypass discharge; steam is fed into the argon main pipeline through a fifth valve (105) and a second flowmeter (402) to be mixed with argon gas at a preset ratio; a steam temperature is measured by a third thermocouple (203); a gas main pipeline is connected to a vacuum pump (4) via a sixth valve (106); an inlet (6) of a heating quenching device is connected to a mixed gas pipeline via a static gas mixer (5) and a seventh valve (107); mixed gas in the heating quenching device is uniformly mixed by the static gas mixer (5); the mixed gas pipeline is equipped with heating wires for temperature control of the mixed gas; the mixed gas pipeline is also equipped with a second pressure sensor (302) and a fourth thermocouple (204); the heating quenching device comprises an infrared radiation furnace (12), a constant-temperature water tank (10), a high-temperature resistant hose (9), a quartz glass tube (13), a sealing ring (20), a quenching quartz glass tube (8), an upper rail slider fixture (16), a lower rail slider fixture (7), a slide rail bracket (17), and a chiller (11), wherein the mixed gas enters the quartz glass tube (13) within the infrared radiation furnace (12) through the inlet (6); a cladding sample is suspended within an infrared focused heating zone (21) at a center of the quartz glass tube (13); the mixed gas is discharged through an outlet (14) of the heating quenching device; a gold-plated reflective surface within the infrared radiation furnace (12) is cooled via the chiller (11), and a pipeline flow rate of the chiller (11) is monitored by a third flowmeter (403), which is then controlled by an eighth valve (108); a height of the cladding sample within the infrared focused heating zone (21) is adjusted via the upper rail slider fixture (16) on an upper portion of the slide rail bracket (17) to achieve uniform heating; a bottom end of the quartz glass tube (13) is opened or closed by the sealing ring (20) located at the bottom end; the lower rail slider fixture (7) at a lower portion of the slide rail bracket (17) clamps the quenching quartz glass tube (8), thereby performing vertical movement of the quenching quartz glass tube (8) within the quartz glass tube (13) for rapid quenching after high-temperature oxidation testing of the cladding sample; a heating temperature of the cladding sample is collected via a fifth thermocouple (205) which is fast in response and exposed, and a heating temperature sequence of the cladding sample is collected through a data acquisition system (18) connected to the exposed fifth thermocouple (205); an infrared radiation furnace temperature control system (19) is connected to the infrared radiation furnace (12) for temperature control of the cladding sample during high-temperature steam oxidation testing.

2. The experimental system, as recited in claim 1, wherein the infrared radiation furnace (12) employs four high-power tungsten filament infrared lamps as heat sources, and heating elements of the infrared lamps are sealed within quartz glass; a stainless steel surface is process with gold plating for reflection and focusing of short-wave infrared radiation; the quartz glass tube (13) is located at a center of the infrared radiation furnace.

3. The experimental system, as recited in claim 1, wherein the slide rail bracket (17) automatically controls vertical movement of the upper rail slider fixture (16) and the lower rail slider fixture (7).

4. The experimental system, as recited in claim 1, wherein constant-temperature water is provided by the constant-temperature water tank (10); water is exchanged between the quenching quartz glass tube (8) and the constant-temperature water tank (10) via the high-temperature resistant hose (9) for temperature control and movement; rapid quenching of the cladding sample after the high-temperature oxidation testing is achieved through automated control of rapid movement of the quenching quartz tube (8).

5. The experimental system, as recited in claim 1, wherein the infrared radiation furnace temperature control system (19) employs a PID (Proportion Integral Differential) algorithm to maintain a constant heating rate, thereby achieving temperature control of the cladding sample during the high-temperature steam oxidation testing.

6. The experimental system, as recited in claim 1, wherein the infrared radiation furnace (12) is capable of heating to 1400° C. with a heating rate exceeding 100° C./s under steam conditions.

7. The experimental system, as recited in claim 1, wherein the data acquisition system (18) comprises a data acquisition card, a measurement module, a signal conditioner and a computer-driven software module, wherein the data acquisition card is connected to the fifth thermocouple (205) via a junction box.

8. The experimental system, as recited in claim 1, wherein the cladding sample is suspended by a platinum-rhodium wire (15) within the infrared focused heating zone (21) at the center of the quartz glass tube (13); wherein the height of the cladding sample within the infrared focused heating zone (21) is adjusted by clamping the platinum-rhodium wire (15) with the upper rail slider fixture (16) on the upper portion of the slide rail bracket (17), thereby achieving uniform heating.

9. An experimental method for high-temperature oxidation and quenching of cladding materials under reactor severe accident, comprising steps of: performing high-temperature oxidation testing on the cladding materials in a steam environment, then performing rapid quenching to obtain mechanical properties of the cladding materials; wherein the experimental method comprises specific steps of: before testing, keeping all valves closed; using a high-precision electronic balance to measure a mass of the cladding sample multiple times and calculating an average value; opening a third valve (103) to introduce deionized water into a steam generator (3) until a preset water level is reached, and then closing the third valve (103); opening a sixth valve (106) and a vacuum pump (4) to evacuate an experimental pipeline, then closing the sixth valve (106); supplying argon gas from a first argon cylinder for testing, with a second argon cylinder (2) serving as a backup; opening a first valve (101) to introduce the argon gas for purging air from the experimental pipeline and from a quartz glass tube (13); opening a fifth valve (105) to feed steam generated by the steam generator (3) into a main argon gas pipeline; activating a static gas mixer (5) and opening a seventh valve (107) to uniformly mix the argon gas and the steam; determining a steam temperature in a mixed gas pipeline by adjusting heating wires and monitoring a temperature sensed by a fourth thermocouple (204); closing a sealing ring (20), so that mixed gas formed by the argon gas and the steam flows upwards through the quartz glass tube (13) and exits through an outlet (14); connecting the cladding sample to an upper rail slider fixture (16) using a platinum-rhodium wire (15), and activating the upper rail slider fixture (16) on a slide rail bracket (17) to move the cladding sample to a bottom of an infrared focused heating zone (21); activating the infrared radiation furnace (12) for heating with a preset heating rate and a target temperature; activating a chiller (11) to cool a stainless steel gold-plated reflective wall of the infrared radiation furnace (12); activating a data acquisition system (18) to collect temperature information of the cladding sample using a fifth thermocouple (205) which is fast in response and exposed, and transmitting the temperature information to an infrared radiation furnace temperature control system (19), thereby controlling a heating rate and a heating temperature of the infrared radiation furnace; just before high-temperature steam oxidation ends, opening the sealing ring (20) at a bottom end of the quartz glass tube (13), and activating a lower rail slider fixture (7) of the slide rail bracket (17) to move a quenching quartz glass tube (8) to the bottom of the infrared focused heating zone (21); at an instant the high-temperature steam oxidation ends, deactivating the infrared radiation furnace (12) while simultaneously activating the lower rail slider fixture (7) to lift the quenching quartz glass tube (8), thereby rapidly quenching the cladding sample after the high-temperature oxidation testing; then activating the upper rail slider fixture (16) of the slide rail bracket (17) to lift and remove the cladding sample, and sequentially closing all pipeline valves and deactivating all experimental instruments;
after testing, measuring the mass of the tested cladding sample using the high-precision electronic balance and calculating the average value; preparing a cross-sectional sample from the cladding sample using a metallographic preparation material, and characterizing oxidation behavior via EDS (Energy Dispersive Spectroscopy), SEM (Scanning Electron Microscopy), or TEM (Transmission Electron Microscopy); subjecting the cladding sample to circumferential compression testing at a preset displacement rate using a circumferential compression testing machine, so as to obtain a stress-strain curve of the cladding sample after quenching, thereby obtaining an offset strain of the cladding sample to characterize the mechanical properties.
